(19) United States
(12) Patent Application Publication
(10) Pub. No.: US 2019/0200892 A1
(43) Pub. Date: Jul. 4, 2019

(54) BIOFLUID SENSING DEVICES WITH INTEGRATIVE EAB BIOSENSORS

(71) Applicant: Eccrine Systems, Inc., Cincinnati, OH (US)

(72) Inventors: Robert Beech, Cincinnati, OH (US); Gavi Begtrup, Cincinnati, OH (US); Jason Heikenfeld, Cincinnati, OH (US); Jacob A Bertrand, Norwood, OH (US)

(73) Assignee: Eccrine Systems, Inc., Cincinnati, OH (US)

(21) Appl. No.: 16/323,705

(22) PCT Filed: Aug. 8, 2017
(86) PCT No.: PCT/US17/45926

§ 371 Date: Feb. 6, 2019

Related U.S. Application Data
(60) Provisional application No. 62/371,902, filed on Aug. 8, 2016.

(51) Int. Cl. A61B 5/053; A61B 10/00; A61B 5/145; A61B 5/1468; G01N 27/327

(52) U.S. Cl. CPC A61B 5/053; A61B 10/0064; A61B 2560/0462; A61B 5/1468; G01N 27/3277; A61B 5/14517

(57) ABSTRACT

The disclosed invention includes integrative electrochemical aptamer-based sensors for use in wearable biofluid sensing devices. The disclosed integrative EAB sensors are configured to detect very low concentrations of target analytes in a sweat or biofluid sample by aggregating signals from individual sensing elements over time until a signal threshold is reached. Signal aggregation is accomplished through various retention structures that extend the time sensing elements retain target analyte molecules. Embodiments include attaching complementary primers and functional groups to the aptamer, covering such retention structures with blockers until analyte capture, or coating the sensor electrode with a hydrophilic and hydrophobic monolayer. The invention also includes methods of using the disclosed integrative sensors. Some embodiments of the disclosed method include tracking time to signal threshold to estimate analyte concentration.

### CROSS-REFERENCE TO RELATED APPLICATIONS

This application claims priority to PCT/US17/45926, filed Aug. 8, 2017, and U.S. Provisional Application No. 62/371,902, filed Aug. 8, 2016, and has specification that builds upon PCT/US17/23399, filed Apr. 21, 2017, the disclosures of which are hereby incorporated herein by reference in their entirety.

### BACKGROUND OF THE INVENTION

Despite the many ergonomic advantages of perspiration (sweat) compared to other biofluids (particularly in “wearable” devices), sweat remains an underutilized source of biomarker analytes compared to the established biofluids: blood, urine, and saliva. Upon closer comparison to other non-invasive biofluids, the advantages may even extend beyond ergonomics: sweat might provide superior analyte information. A number of challenges, however, have historically kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in “wearable” sweat sampling and sensing devices has resolved several of the historical challenges. However, this recent progress has also been limited to high concentration analytes (μM to mM) sampled at high sweat rates (>1 nL/min/gland) found in, for example athletic applications. Progress will be much more challenging as sweat biosensing moves towards detection of large, low concentration analytes (nM to pM and lower).

In particular, many known sensor technologies for detecting larger molecules are ill-suited for use in wearable sweat sensing, which requires sensors that permit continuous use on a wearer's skin. This means that sensor modalities that require complex microfluidic manipulation, the addition of reagents, the use of limited shelf-life components, such as antibodies, or sensors that are designed for a single use will not be sufficient for sweat sensing. Electrochemical aptamer-based (“EAB”) sensor technology, such as is disclosed in U.S. Pat. Nos. 7,803,542 and 8,003,374, presents a stable, reliable bioelectric sensor that is sensitive to the target analyte in sweat, while being capable of multiple analyte capture events during the sensor lifespan.

As disclosed in PCT/US17/23399, EAB sensors for use in continuous sweat sensing are configured to provide stable sensor responses with a life cycle extensive enough for multiple analyte binding and release cycles. Such sensors include a plurality of individual aptamer sensing elements 110, as depicted in FIG. 1A, which can repeatedly detect the presence of a molecular target by capturing and releasing target analytes as they come in contact with the aptamer. The sensing element 110 includes an analyte capture complex 112, which includes an aptamer 140 that is selected to preferentially bind to a target analyte 160, a redox moiety 150, such as methylene blue, bound to a first end of the aptamer, and a first nucleotide primer 142 that is bound to the other end of the aptamer. In other embodiments (not shown), the first end of the aptamer may be bonded to a second nucleotide primer, which is then bonded to the redox moiety. The analyte capture complex 112 is covalently bonded via the first primer 142 to a thiol 120 or other suitable anchoring molecule or complex, which is in turn covalently bonded to an electrode 130. An analyte capture complex 112 may also be bound to the electrode by means of an EDTA strain to improve adhesion in difficult environments. When the analyte capture complex 112 is in a first configuration, as depicted in FIG. 1A, the redox moiety 150 is in a first position relative to the electrode 130. Upon interrogation by a square wave voltammetry signal, the electrode produces a first electrical signal eT_{A} indicative of this first redox position.

With reference to FIG. 1B, when the aptamer 140 interacts with the target analyte 160, the first configuration is disrupted, and a second configuration is formed. The formation of the second configuration moves the redox moiety 150 into a second position relative to the electrode 130, which then produces a second electrical signal eT_{B} upon interrogation, that is distinguishable from the first electrical signal eT_{A}. After a recovery interval of seconds or minutes, the aptamer 140 releases the target analyte 160. Following this release, the aptamer 140 returns to the first configuration, and the electrode 130 returns to registering the corresponding first electrical signal eT_{A}.

EAB sensors, as depicted in FIGS. 1A and 1B, are successful in measuring larger analytes, or higher concentration analytes. However, many analytes are present in sweat at very low concentrations. These low analyte concentrations can significantly impact an EAB sensor's ability to provide reliable, continuous sensing. Concentration ranges for potential EAB sensor analytes span from mM for lactate, to μM for luteinizing hormone, to nM for cortisol, to pM and even fM ranges for larger proteins. When target analyte concentrations are lower, the aptamers in EAB sensors will naturally have fewer capture opportunities, requiring greater sensitivity to ensure that the reduced capture opportunities are fully exploited.

Additionally, EAB sensors that target small analytes are inherently less stable and, thereby, produce less reliable signals, than sensors targeting larger analytes. Target analytes for sweat sensor applications as contemplated herein may range in size from ˜300 Da for hormones to ˜15 kDa for microRNA molecules to ˜600 kDa for larger proteins to ˜1000 kDa for the largest proteins. Other factors being equal, aptamers will generally develop stronger bonds to larger molecules because of the greater number of bonding sites available on such molecules. Furthermore, sweat sample composition variabilities that tend to alter bonding strength (such as pH and salinity) generally have a greater effect on smaller molecule sensors than on larger molecule sensors. Aptamer sensing elements configured to detect small molecules will tend to have shorter recovery intervals (i.e. seconds or faster), due to the low number of binding sites. The instability created by small-sized analytes is compounded as the analyte concentration in a biofluid sample decreases.

Therefore, for smaller analytes, or analytes appearing in low concentrations in a biofluid such as sweat, an EAB sensor as described above may be unable to accurately perform continuous sensing. Too few target analytes may be present in a sweat sample, such that during any given recovery interval, not enough analytes will bond with an aptamer to meet the signal threshold. Following the recovery interval, bound analytes will release back into solution before another chronologically assured sweat sample can be measured. In such a scenario, while the target analyte is present in the sweat sample, the sensing device will not be able to provide an accurate measurement of the analyte's presence, much less a reliable concentration value.

Accordingly, for small analyte and low analyte concentration applications, it is desirable to have an EAB sensor that can provide a qualitative “yes/no” measurement for the presence or absence of a target analyte. In particular, it is desirable to have EAB sensing devices and methods that can accurately assess a small or low concentration analyte's presence in a sweat sample by aggregating analyte captures over an extended period of time.

The disclosed invention can improve its performance for biofluid sensing by contextualizing data generated by the device with relevant external information. Such contextualization may include collecting the biofluid sensor data generated by biofluid sensing devices and correlating that data with relevant outside information, such as the time, date, medications, medical condition, the proximity to significant health events or stressors, age, sex, health history, or other relevant information. The sweat sensor data monitored by the user includes real-time data, trend data, or may also include aggregated sweat sensor data drawn from the system database and correlated to a particular user, a user profile (such as age, sex or fitness level), weather condition, activity, combined analyte profile, or other relevant metric. Such predictive capability can be enhanced by using correlated aggregated data, which would allow the user to compare an individual's historical analyte and external data profiles to a real-time situation as it progresses, or even to compare thousands of similar analyte and external data profiles from other individuals to the real-time situation. Biofluid sensor data may also be used to identify wearers that are in need of additional monitoring or screening.

Many of the other challenges to successful sweat sensor development can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat to sensors and sample preparing or concentrating subsystems.

### SUMMARY OF THE INVENTION

The disclosed invention includes integrative electrochemical aptamer based sensors for use in wearable biofluid sensing devices. The disclosed integrative EAB sensors are configured to detect very low concentrations of target analytes in a sweat or biofluid sample by aggregating signals from individual sensing elements over time until a signal threshold is reached. Signal aggregation is accomplished through various retention structures that extend the time sensing elements retain target analyte molecules. Embodiments include attaching complementary primers and functional groups to the aptamer, covering such retention structures with blockers until analyte capture, and coating the sensor electrode with a hydrophilic and hydrophobic monolayer. The invention also includes methods of using the disclosed integrative sensors. Some embodiments of the disclosed method include tracking time to signal threshold to develop an analyte concentration estimate.

### BRIEF DESCRIPTION OF THE DRAWINGS

The objects and advantages of the present invention will be further appreciated in light of the following detailed descriptions and drawings in which:

FIGS. 1A and 1B are representations of a previously-disclosed aptamer sensing element before and after analyte capture;

FIGS. 2A and 2B are diagrammatic views of a first exemplary embodiment of an integrative aptamer sensing element before and after analyte capture;

FIGS. 3A & 3B are diagrammatic views of another exemplary embodiment of an integrative aptamer sensing element before and after analyte capture;

FIGS. 4A and 4B are diagrammatic views of at least a portion of a biofluid sensing device with an integrative aptamer sensing element, shown before and after analyte capture;

FIGS. 5A and 5B are diagrammatic views of at least a portion of another exemplary embodiment of the disclosed invention including an integrative docked aptamer sensing element, shown before and after analyte capture;

FIGS. 6A and 6B are diagrammatic views of at least a portion of an exemplary integrative EAB sensor depicting multiple aptamer sensing elements; and

FIG. 7 is a schematic depiction of an exemplary biosensing device with at least one integrative EAB sensor.

### DEFINITIONS

“Continuous monitoring” means the capability of a device to provide at least one measurement of sweat determined by a continuous or multiple collection and sensing of that measurement or to provide a plurality of measurements of sweat over time.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, “biofluid” may mean any human biofluid, including, without limitation, sweat, interstitial fluid, blood, plasma, serum, tears, and saliva. A biofluid may be diluted with water or other solvents inside a device because the term biofluid refers to the state of the fluid as it emerges from the body.

“Sweat” or “sweat biofluid” means a fluid that is comprised mainly of interstitial fluid or sweat as it emerges from the skin. For example, a fluid that is 45% interstitial fluid, 45% sweat, and 10% blood is a sweat biofluid as used herein. For example, a fluid that is 20% interstitial fluid, 20% sweat, and 60% blood is not a sweat biofluid as used herein.

“Sweat sampling rate” is the effective rate at which new sweat or sweat solutes, originating from the sweat gland or from skin or tissue, reaches a sensor which measures a property of sweat or its solutes. Sweat sampling rate, in some cases, can be far more complex than just sweat generation rate.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Measured” can imply an exact or precise quantitative measurement and can include broader meanings such as, for example, measuring a relative amount of change of something. Measured can also imply a binary measurement, such as ‘yes’ or ‘no’ type measurements.

“Microfluidic components” are channels in polymer, textiles, paper, or other components known in the art of microfluidics for guiding fluid movement or at least partial containment of a fluid.

“Flow rate sensing component”, is any component or components which measure the flow rate of sweat in at least one portion of a sweat sensing or collecting device.

“Analyte” means a substance, molecule, ion, or other material that is measured by a sweat sensing device.

“Biofluid sensor data” means all information collected by biofluid sensing device sensor(s) and communicated to a user or a data aggregation location.

“Correlated aggregated biofluid sensor data” means biofluid sensor data that has been collected in a data aggregation location and correlated with outside information such as time, temperature, weather, location, user profile, other biofluid sensor data, or any other relevant data.

“Analyte capture complex” means an aptamer, or other suitable molecules or complexes, such as proteins, polymers, molecularly imprinted polymers, polypeptides, and glycans, that experience a conformation change in the presence of a target analyte, and are capable of being used in an EAB sensor. Such molecules or complexes can be modified by the addition of one or more primers comprised of nucleotide bases.

“Aptamer sensing element” means an analyte capture complex that is functionalized to operate in conjunction with an electrode to detect the presence of a target analyte. Such functionalization may include tagging the aptamer with a redox moiety, or attaching thiol binding molecules, docking structures, or other components to the aptamer. Multiple aptamer sensing elements functionalized on an electrode comprise an EAB sensor.

“Docked aptamer EAB sensor” means an EAB sensor that employs docking strategies to connect analyte capture complexes with the sensor electrode, as disclosed in U.S. Provisional No. 62/523,835, filed Jun. 23, 2017, which is hereby incorporated by reference in its entirety herein.

“Integrative EAB sensor” means an aptamer-based biosensor that is configured with multiple aptamer sensing elements that produce a sustained signal indicating target analyte capture, and which signal can be added to the signals of other such sensing elements, so that over time a signal threshold may be reached that indicates the presence of the target analyte.

“Recovery interval” means the time required for an aptamer sensing element to release a target analyte back into solution and return to its signal-off position.

“Signal threshold” means the combined strength of signal-on indications produced by a plurality of integrative aptamer sensing elements that indicates the presence of a target analyte.

“Time-to-threshold” means the amount of time required for an integrative EAB sensor to reach signal threshold. Such time may be calculated from the initiation of device use, the initiation of sweating, a sensor regeneration time, or other suitable starting point.

“Preferential energy state” means a relatively stable configuration of an analyte capture complex, typically requiring an energy input to allow the complex to change to a different configuration.

### DETAILED DESCRIPTION OF THE INVENTION

Integrative EAB sensors are described herein for use within a biofluid sensing device. The EAB sensors include a plurality of aptamer sensing elements that are selected to capture a target analyte in a biofluid, such as sweat. Rather than releasing the analyte and returning to a “signal-off” configuration following the typical aptamer recovery interval, as with the aptamer sensing elements described above, the aptamer sensing elements in the disclosed invention remain in a “signal-on” configuration for an extended period of time (e.g., several minutes, or hours), while sweat samples continuously or periodically flow past the sensors. Retaining the aptamer sensing elements in an analyte capture configuration for an extended period of time enables the “capture” signals from the sensing elements to be aggregated over the extended time period. The extended period enables sufficient analyte captures to occur for the aggregated signal to reach the signal threshold indicative of the target analyte's presence in the sweat samples.

To select suitable aptamers for the integrative EAB sensors, various methods known in the art of aptamer selection may be used, including, for example, Systematic Evolution of Ligands by Exponential Enrichment (“SELEX”) techniques. Using such techniques, an aptamer for binding a target analyte is selected to reliably detect the analyte at very low concentration levels. The aptamer is also chosen for its affinity to the target molecule, such that the analyte will be reliably captured, giving the integrative EAB sensor the desired selectivity and specificity. For many target analytes, however, the selected aptamer will not possess a recovery interval that is long enough to allow for a detectable capture signal from the electrode. For such analytes, therefore, other structural elements may be incorporated into an EAB sensor to retain an aptamer sensing element in a preferential energy state for an extended time period upon analyte capture.

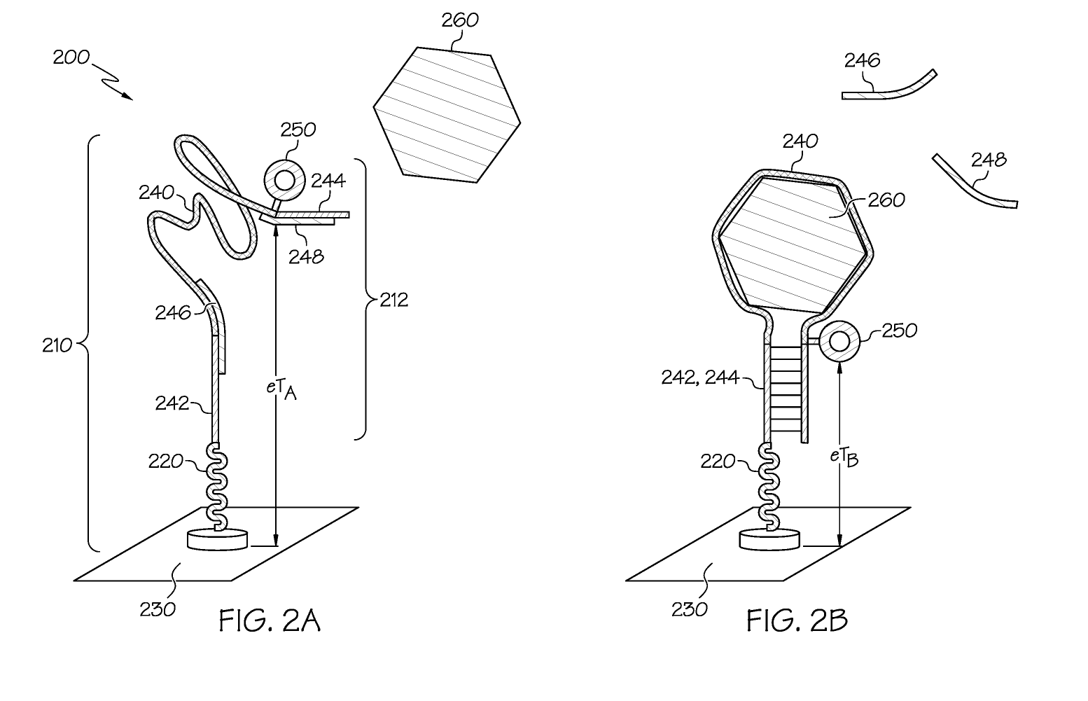

For example, with reference to FIG. 2A, in a first exemplary embodiment a biofluid sensing device 200 includes a plurality of aptamer sensing elements 210, each element including an analyte capture complex 212 immobilized on an electrode 230. While the figures depict, and the discussion focuses on, a single aptamer sensing element, EAB sensors in the exemplary embodiments described herein will include a large number of aptamer sensing elements 210 (thousands, millions, or billions of individual sensing elements, having an upper limit of 10¹⁴/cm²) attached to the electrode. The sensing element 210 may be attached to the electrode 230 by covalently bonding a first end to a thiol, which is then in turn covalently bonded to the electrode. The electrode 230 may be comprised of gold or another suitable conductive material. Aptamer sensing element 210 further includes an aptamer 240, a redox moiety 250 such as, for example, methylene blue, and a complementary pair of nucleotide primers 242, 244 bonded to opposite ends of the aptamer. One or more oligonucleotide blocker strands or sections 246, 248 are bound to analyte capture complex 212 to prevent the complementary primers 242, 244 from binding prior to analyte capture. When no target analyte is present, the analyte capture complex assumes a first configuration, and spacing between the redox 250 and electrode 230, which upon interrogation by a square wave voltammetry signal, causes the electrode to produce a first electrical signal, eT_{A}.

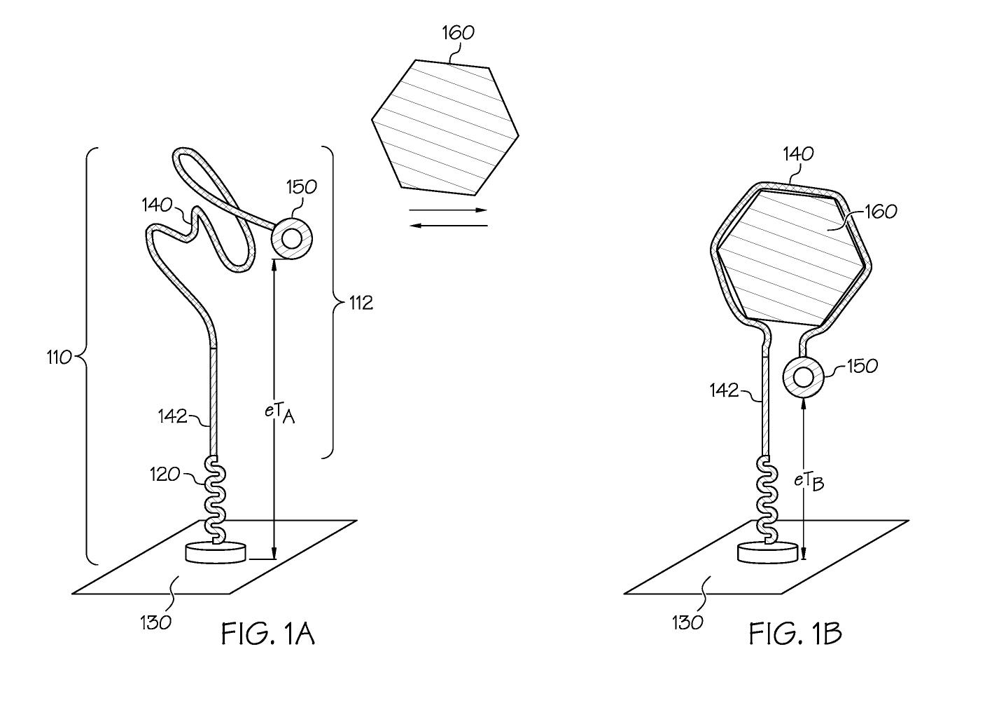

With reference to FIG. 2B, when the aptamer 240 interacts with or “captures” the target analyte 260, the first configuration is disrupted, and a second configuration is formed. The disruption of the first aptamer configuration causes the blocker strands 246, 248 to break free from the analyte capture complex 212 and move away. The formation of the second configuration also moves the redox moiety 250 into a second position relative to the electrode 230. In this second position, a second electrical signal eT_{B}, that is distinguishable from the first electrical signal eT_{A}, is produced upon interrogation of the electrode 230. With the blocker strands 246, 248 separated from aptamer sensing element 210, the complementary primers 242, 244 bind together, creating a favorable energy state for the analyte capture complex 212 in the second configuration. The favorable energy state holds the analyte capture complex 212 in the second configuration, to thereby generate the second, capture signal eT_{B} for an extended period of time. One or more additional functional groups, or non-native bases, may be added to the linking strands 242, 244 to increase the strength of the bond between the linked strands, and to further prevent release of the analyte 260. Unlike the embodiment depicted in FIGS. 1A and 1B, the loss of the blocker strands 246, 248, and the favorable energy state of the binding between the complementary primers 242, 244, prevents the analyte capture complex 212 from returning to the first configuration. Because the aptamer 240 is locked into the second configuration by the primer bond, the aptamer sensing elements 210 of this embodiment are suitable for a one-time use, rather than multiple capture and release cycles.

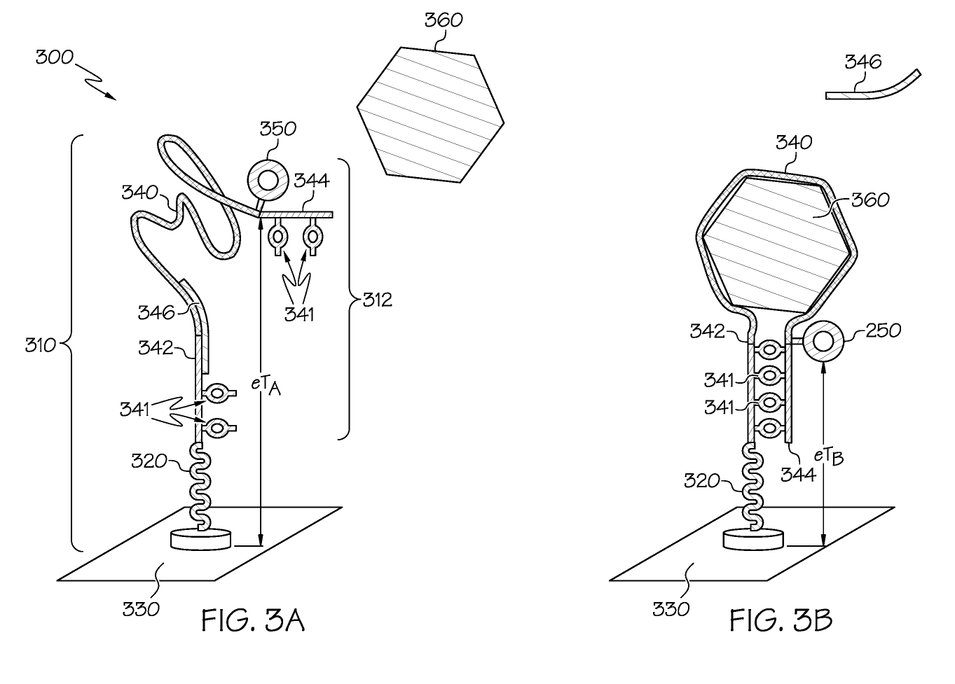

In an alternative embodiment, depicted in FIG. 3A, a biofluid sensing device 300 comprises a plurality of aptamer sensing elements 310, each of the elements including an analyte capture complex 312 with two nucleotide primers 342, 344 on opposite ends of an aptamer 340. The analyte capture complex 312 is covalently bonded via the first primer 342 to a thiol 320, which is in turn covalently bonded to an electrode 330. In this embodiment, the primers 342, 344 serve as a mounting location for a plurality of functional groups, indicated by reference numeral 341. Analyte capture complex 312 also includes one or more blocker sections 346 that prevent the functional groups from interacting prior to analyte capture. The functional groups 341 include, e.g., phenyl groups, carboxylic acids, alkyl chains, pyridinyl rings or other suitable groups that when placed in proximity with each other will interact to maintain that close proximity. When no target analyte is present, the analyte capture complex 312 assumes a first configuration, which has a corresponding first electrical signal eT_{A}.

When the aptamer 340 captures a target analyte 360, the first configuration is disrupted, and a second configuration is formed, causing the blocker 346 to break free from the analyte capture complex 312 and move away. The formation of the second configuration also moves the redox moiety 350 into a second position relative to the electrode 330. This change in redox position causes the electrode 330 to produce a second electrical signal eT_{B}, that is distinguishable from the first electrical signal eT_{A}, when the electrode is interrogated. When the aptamer 340 moves into the second configuration, functional groups 341 on opposite ends of the aptamer are drawn into close proximity with each other. This close proximity between functional groups 341 creates a favorable energy state for the analyte capture complex 312 in the second configuration, retaining the aptamer in the second configuration, and allowing the aptamer sensing element 310 to generate capture signal eT_{B} for a sustained period of time. As with the previous embodiment, the separation of the blocker 346 from aptamer sensing element 310 on capture of analyte 360, and the subsequent bond between functional groups 341, prevents the aptamer 340 from returning to the first configuration. Accordingly, sensing devices of this embodiment are one-time use sensors. Some embodiments may include functional groups attached to the redox moiety (not shown).

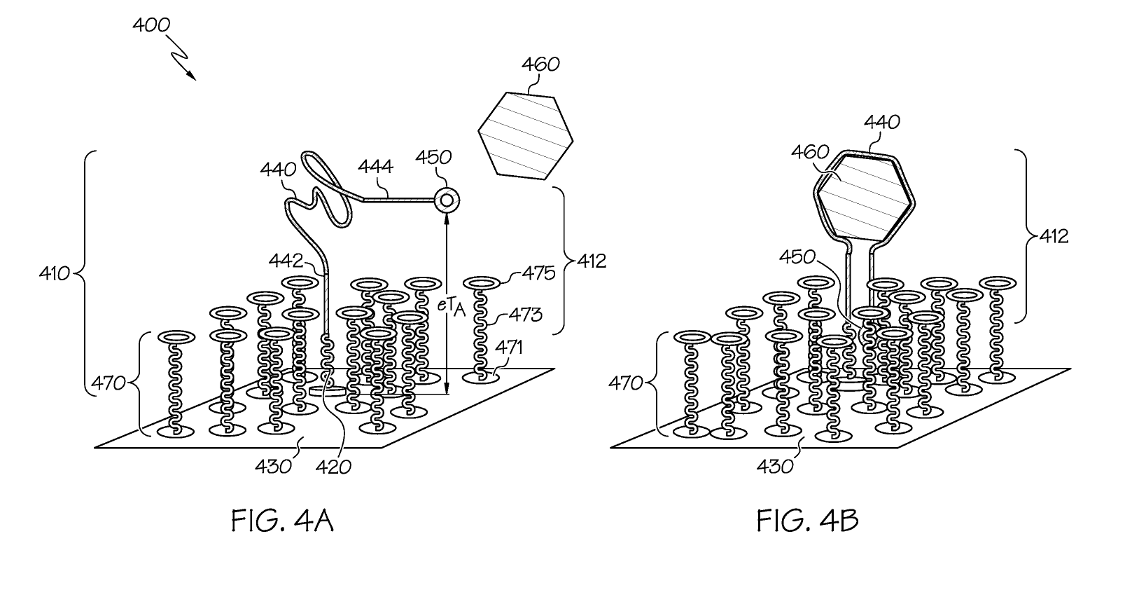

In another alternative embodiment, depicted in FIG. 4A, a biofluid sensing device 400 comprises a plurality of aptamer sensing elements 410, arranged similarly to the embodiments described above. As described above, each of the aptamer sensing elements 410 includes an analyte capture complex 412, having an aptamer 440 that is selected to preferentially bind to a target analyte 460. First and second primers 442, 444 are bonded to opposite ends of the aptamer 440, and a hydrophobic redox moiety 450 is bonded to the second primer 444. The analyte capture complex 412 is covalently bonded via the first primer 442 to a thiol 420, which is in turn covalently bonded to an electrode 430. The electrode 430 is coated with a self-assembled monolayer (SAM) including a plurality of blocking structures 470. Each of the blockers 470 includes a hydrophobic tether 473, which is bonded at a first end to a thiol 471 and at a second, opposite end to a hydrophilic hydroxide group 475. When the analyte capture complex 412 is in a first configuration, depicted in FIG. 4A, the redox moiety 450 is in a first position relative to the electrode 430, and a first electrical signal eT_{A} is produced upon interrogation of the electrode. Because the redox moiety is hydrophobic, the SAM's hydrophilic surface 475 will tend to repel the redox moiety, thereby maintaining the analyte capture complex in the first configuration.

However, when aptamer 440 captures a target analyte 460, the first configuration is disrupted, and a second configuration, depicted in FIG. 4B, is formed. The formation of the second configuration moves the redox moiety 450 into a second, different position relative to the electrode 430. The change in location of redox moiety 450 causes a second, different electrical signal eT_{B} to be produced upon interrogation of the electrode 430. In the second position, the hydrophobic redox moiety 450 is moved into close proximity to the hydrophobic tethers 473, and the hydrophilic DNA sequence of the aptamer 440 into close proximity to the hydrophilic hydroxide group 475. The close proximity between both sets of hydrophobic and hydrophilic molecules creates a preferential energy state for the analyte capture complex 412 in the second configuration. This preferential energy state retains redox moiety 450 in the second configuration beyond the normal recovery interval of the aptamer 440. Thus, the capture signal eT_{B} is produced for a sustained period of time, beyond the normal recovery interval for the aptamer. The sustained signal period allows for additional analyte captures by other analyte capture complexes 412, and the aggregation of additional analyte capture signals eT_{B}. Upon interrogation of electrode 430, the aggregated analyte capture signals can be compared to the signal threshold for the sensor to measure the presence of the target analyte.

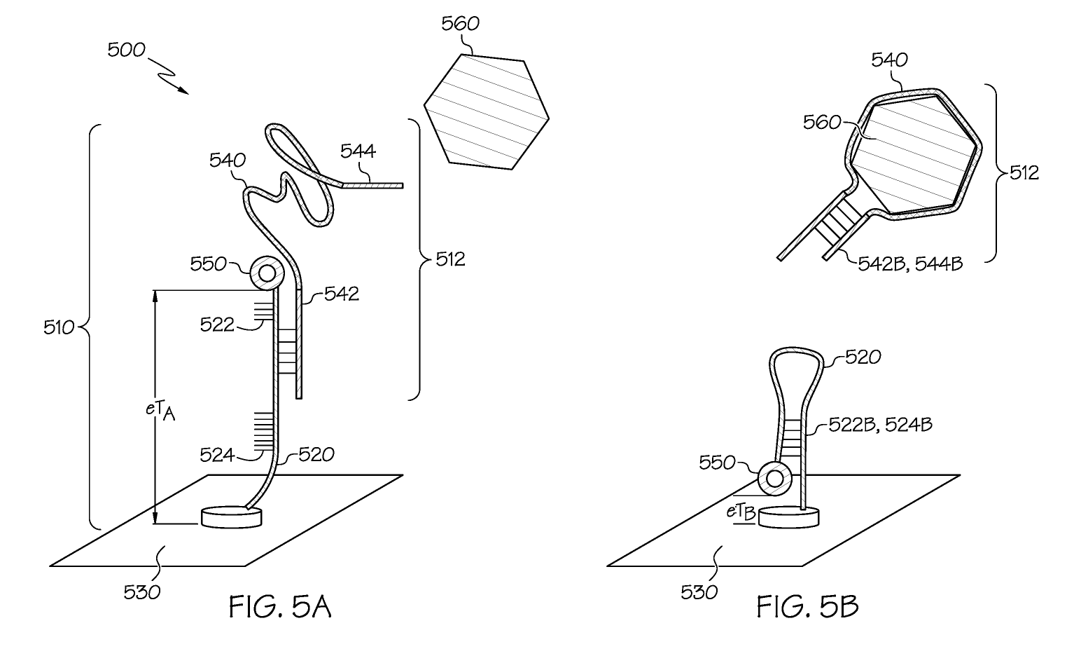

In another alternative embodiment, depicted in FIGS. 5A and 5B, a biofluid sensing device 500 comprises a plurality of aptamer sensing elements 510. Each of the aptamer sensing elements 510 includes an analyte capture complex 512 and a molecular docking structure 520 immobilized on an electrode 530. The dock 520 may be attached to the electrode 530 by covalently bonding a first end to a thiol, which is then in turn covalently bonded to the electrode. The electrode 530 may be comprised of gold or another suitable conductive material. Analyte capture complex 512 includes an aptamer 540 and a first nucleotide primer sequence 542, which is bonded to a first end of the aptamer. A second nucleotide primer 544, which is complementary to the first primer 542, is bonded to the second end of the aptamer 540. The dock 520 includes a 9 to 12 base nucleotide sequence, indicated at 521, that is selected to be complementary with the first primer 542, in order to bond the dock 520 to the analyte capture complex 512. A redox moiety 550 is immobilized on the dock 520, on a second end, opposite the electrode 530. The dock 520 further includes two complementary nucleotide sequences 522, 524. When bound to the analyte capture complex 512 via the complementary nucleotide sequences, the dock 520 has a stiff configuration, allowing the redox moiety 550 to be located at a maximum distance from the electrode 530. As shown in FIG. 5A, this distance is approximately the full length of the dock 520. The distance between the redox moiety 550 and electrode 530 is sufficiently large to prevent most electron transduction, thereby largely preventing redox of the redox moiety in response to potentials applied via electrode 530. In this first configuration, a no or reduced signal eT_{A} is generated from the redox moiety 550 upon interrogation of electrode 530.

In operation, the aptamer sensing element 510 is exposed to a biofluid sample containing a concentration of the target analyte 560. With reference to FIG. 5B, on interaction with the target analyte 560, the aptamer 540 is drawn physically around the analyte 560 to capture the analyte, causing the second primer 544 to move into physical proximity to the first primer 542. The physical proximity of the complementary primers causes the first primer to break free from the docking structure 520 and bind to the second primer, as indicated at 542B, 544B. Upon separation from the dock, the analyte capture complex 512 and captured analyte 560 move away from the dock 520. Once the dock 520 is unbound from the analyte capture complex 512, the dock becomes more flexible, allowing the complementary primers 522, 524 to bind together as indicated at 522B, 524B. The binding of primers 522, 524 causes the dock 520 to move into a second, folded configuration depicted in FIG. 5B. The folding of dock 520 locks the attached redox moiety 550 in a position close to the electrode 530. Interrogation of the electrode 530 following analyte capture will return a detectable signal eT_{B} that differs from the no or reduced signal eT_{A}, due to the proximity of the redox moiety 550 to the electrode.

Each of the embodiments described above allows an aptamer sensing element to capture and retain a target analyte beyond a typical recovery interval for the selected aptamer. In each of the embodiments, a capture signal eT_{B} is produced for a sustained period of time, through multiple sweat sampling intervals. The sustained signal period allows for additional analyte captures by other aptamer sensing elements in the sensor, and the aggregation of all the analyte capture signals. The aggregated signal can be compared to the sensor signal threshold to measure the presence of the target analyte. Accordingly, the devices described herein allow a greater time-to-threshold for an EAB sensor to provide increased measurement accuracy, particularly with small and/or low concentration analytes.

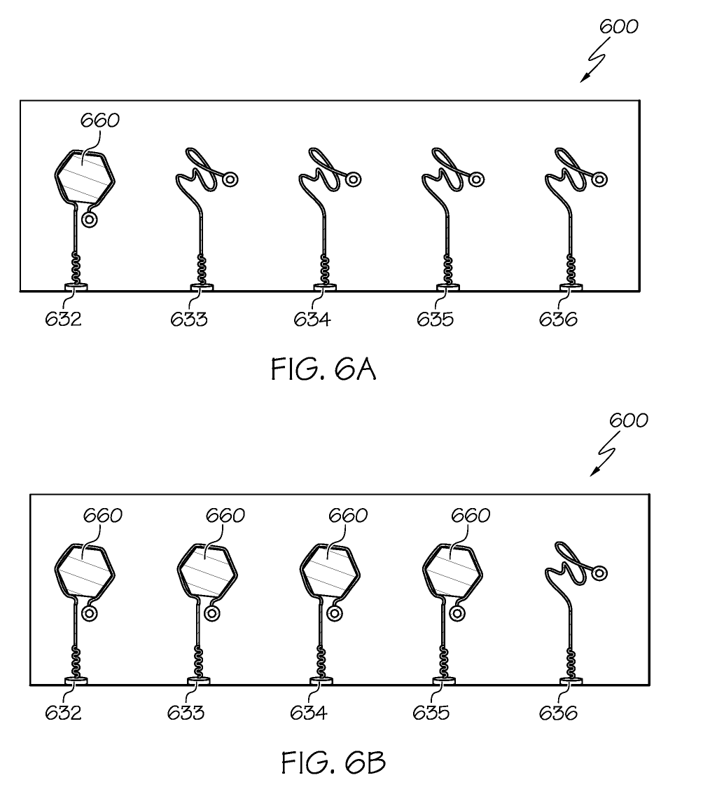

Turning now to FIGS. 6A and 6B, which depict exemplary operation of integrative EAB sensors as described above. As shown in FIG. 6A, a sensor 600 has been exposed to continuous or periodic sweat samples over several hours. However, despite continuous monitoring for an extended period of time, only one aptamer sensing element, indicated at 632, has captured an analyte 660 and is generating a captured signal on interrogation of the electrode. In this scenario, the sensor 600 would not reach the signal threshold and, therefore, would not register detection of the target analyte 660.

With reference to FIG. 6B, in another example the sensor 600 has been exposed to sweat samples for the same duration as the device in FIG. 6A. In this example, however, aptamer sensing elements 632, 633, 634, and 635 have all captured an analyte 660, and are generating a capture signal on interrogation of the electrode. In this example, the individual capture signals from the aptamer sensing elements would be added to allow the sensor 600 to reach the signal threshold, and thereby register detection of the target analyte 660. In some embodiments, the signal threshold may represent an approximate number of molecules of the target analyte. The number of signal-on sensing elements required to reach a signal threshold, or the required strength of the threshold signal, may be determined based on a number of factors, including the amount of certainty required for positive detection, reliability of the sensing elements, sweat sample conditions (salinity, pH), analyte size, or other factors.

For example, with regard to required certainty, EAB sensing devices as described herein may be designed to produce a predictive value for the desired application, which balances false positive indications and false negative indications. Some applications, such as a sweat sensing device used to screen the general population for a heart condition, may require very low false positive indications, and therefore would need to have a higher signal threshold, representing greater certainty of analyte presence. Other applications, such as preliminary screening for lead exposure in an at-risk population, may not require such high certainty, and may use a lower signal threshold. In other cases, an aptamer sensing element may have an aptamer that relatively weakly binds the target analyte, or the particular sweat sample may have challenging pH or salinity characteristics, or the target analyte may be very small. In each of these cases, the signal threshold would need to be relatively higher than in the converse case, all other factors being equal.

In other embodiments, the disclosed invention may also be configured to derive a quantitative measurement of target analytes at low concentrations. In one embodiment, the device may simply track the time required to reach the signal threshold, or time-to-threshold. If the device is placed on skin and subsequently detects the signal threshold within a few minutes, the device can infer that the analyte exists in higher sweat concentrations than if the time-to-threshold were several hours. For example, if a device configured to determine the presence of inflammation by detecting cytokines takes 5 hours to reach signal threshold, the device may recommend that no action be taken. However, if the device reaches signal threshold after only 2 hours, the device may recommend further necessary action. Such time-to-threshold calculations would not be relevant for applications requiring only qualitative measurements, such as a device monitoring for the presence of Ebolavirus or other exogenous molecules that under normal conditions would not be present in sweat. Similarly, sweat rate contributes to concentration estimates based on time-to-threshold, since an analyte that reaches signal threshold at a lower sweat rate may be interpreted as having a higher concentration than an analyte that reaches signal threshold at the same time-to-threshold, but at a higher sweat rate, other factors being equal.

By their potential to recover, or to register trend information, integrative EAB sensors represent a significant improvement over current lateral flow assay technologies. For some applications, an integrative EAB sensor can provide trend information by examining time-to-threshold for a follow-on signal threshold. For example, an integrative sensor has a first signal threshold, and is configured with a second, higher, signal threshold. The time required for the sensor to reach this second threshold could be used to indicate trend information. MCAS embodiments could also return to a signal-off state, which would indicate that the concentration trend was generally downward, and could indicate that remediation techniques were effective, for instance that water intake relieved an indicated state of dehydration.

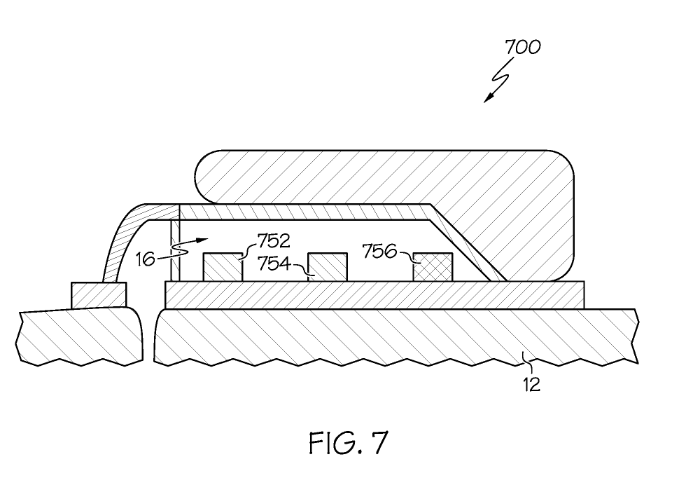

In some embodiments, a biofluid sensing device for use with the disclosed integrative EAB sensor is configured to estimate a biofluid concentration of a target analyte. FIG. 7 depicts an example biofluid sensing device 700 that includes at least one integrative EAB sensor 752, and includes the capability to measure a sweat generation rate and the time required to reach a signal threshold. Such an example device includes a plurality of aptamer sensing elements as described in the previous embodiments, and at least one flow rate sensing component or sensor 754 for measuring sweat flow 16 through the device. The sweat flow sensor 754 may be, for example, a galvanic skin response (GSR) sensor. Device 700 may also include a third sensor 756 which may be, for example, a sweat conductivity sensor, a skin impedance sensor, a micro-thermal flow sensor, or an ion-selective electrode sensor for at least one of Na⁺ or Cl⁻. In use, the sensing device 700 may detect sweat onset and cessation with the GSR sensor 754, and sweat flow rate with the additional sensor 756. By tracking the sweat flow rate, and the time-to-threshold for the sensor 752, the device can use the disclosed integrative EAB sensor modality to calculate the analyte concentration in the sweat flow.

Biofluid sensing devices with integrative EAB sensors, as described above, may be further configured for improved low analyte concentration detection. For example, a sensor may be electromagnetically shielded to reduce the effects of electrical noise, thereby improving sensor sensitivity. Alternately, an EAB sensor may be placed downstream of a sample pre-concentration channel, which would remove water and saline ions or molecules from the sweat sample, to buffer the sample, and increase relative analyte concentration. Similarly, an aptamer sensing element may be surrounded by neutral pH fluid to improve sensitivity for small and low concentration analytes.

Some embodiments benefit from additional techniques to extend the recovery interval for aptamer sensing elements. For example, aptamer sensing elements could be periodically exposed to a light source that polymerizes the sensing elements and captured analytes in their bound state, thus extending the recovery interval. Accordingly, the structures described herein allow a greater time period for creation of analyte capture signals to account for low concentration analytes. Also, the extended capture period enables aptamers to retain small molecules longer than the typical recovery interval, allowing a combined signal to be created from a higher number of captured analytes.

In some sensing device applications, it may desirable to have an EAB sensor in which the aptamer sensing elements can be regenerated after a signal threshold is reached, to allow for multiple capture and release cycles. Regeneration of the aptamer sensing elements may be accomplished, for example, by placing a heating component in proximity to the sensor, which would cause the target analytes to detach from the sensing elements and return to solution. Regeneration of the sensing elements may also be accomplished by introducing a buffering fluid to the EAB sensor such as, for example, by means of a small-volume fluid reservoir containing neutral pH fluid. The buffering fluid would allow the analytes to be released from the aptamers and returned to the solution. Various other solvents (such as alcohols, ethers, aldehydes, halogenate molecules) as known in the art, may also be used in conjunction with, or instead of, water. Other solutions may include molecules (such as a surfactant or a detergent) that cause the analytes to release and return to solution.

While several exemplary embodiments have been described herein for enabling a greater time period for creation of analyte capture signals for measuring low concentration analytes, it is anticipated that other materials, elements and configurations may also be used, provided the alternative materials, elements and/or configurations provide chronological assurance and accurate measurements of the analyte. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

1. An electrochemical aptamer-based (“EAB”) sensor for use in a wearable biofluid sensing device, the sensor comprising:
a plurality of aptamer sensing elements, each aptamer sensing element having a selected aptamer sequence capable of interacting with a target analyte in a biofluid, each aptamer sensing element forming a first configuration before analyte capture and a second configuration after analyte capture;
a redox moiety;
one or more linkers, wherein the one or more linkers attach the selected aptamer sequence to one or more of the following: the redox moiety, and the electrode;
an electrode operative in conjunction with the plurality of aptamer sensing elements to produce a variable signal depending upon the configuration of each aptamer sensing element, wherein upon interrogation of the electrode, each aptamer sensing element produces a first signal when the aptamer sensing element is in the first configuration, and a second signal when the aptamer sensing element is in the second configuration; and
a plurality of retaining structures for retaining a subset of the plurality of aptamer sensing elements in the second configuration beyond a recovery interval to allow the electrode to produce an aggregated signal indicative of the presence of the target analyte.

2. The EAB sensor of claim 1, wherein the retaining structures further comprise a first primer attached to a first end of the aptamer, and a second primer attached to a second end of the selected aptamer sequence, wherein the first primer is complementary with the second primer, and whereupon interaction of the selected aptamer sequence with the target analyte, the primers bind together to retain the aptamer sensing element in the second configuration beyond the recovery interval.

3. The EAB sensor of claim 1, wherein the retaining structures further comprise one or more blockers, the one or more blockers being bound to each aptamer sensing element in a position to prevent the primers from binding together prior to analyte capture.

4. The EAB sensor of claim 1, wherein the retaining structures further comprise a plurality of functional groups, whereupon interaction of the plurality of aptamer sensing elements with the target analyte, a subset of the plurality of functional groups interact with each other to retain the aptamer sensing element in the second configuration beyond the recovery interval.

5. The EAB sensor of claim 1, further comprising a self-assembled monolayer of hydrophilic and hydrophobic structures attached to a surface of the electrode, wherein the hydrophilic structures form a surface for repelling the redox moiety prior to analyte capture by the aptamer sensing element, and whereupon interaction of the selected aptamer sequence with the target analyte, the hydrophobic structures attract the redox moiety and the hydrophilic structures attract the selected aptamer sequence to retain the aptamer sensing element in the second configuration beyond the recovery interval.

6. A method of using the electrochemical aptamer-based (“EAB”) sensor of claim 1 to perform integrative analyte sensing in a biofluid, the method comprising:
providing a plurality of aptamer sensing elements in the EAB sensor, wherein the plurality of aptamer sensing elements are attached to an electrode in a first configuration, wherein the first configuration corresponds to a first signal;
exposing the plurality of aptamer sensing elements to a biofluid potentially containing a target analyte;
producing a conformational change in a subset of the plurality of aptamer sensing elements to a second configuration on interaction with the target analyte;
retaining the subset of the plurality of aptamer sensing elements in the second configuration for a period, wherein the period is of sufficient length to generate a signal;
generating an aggregated signal from plurality of aptamer sensing elements; and
comparing the aggregated signal to one or more signal thresholds to develop a measurement of the target analyte.

7. The method of claim 6, wherein the step of retaining the subset of the plurality of aptamer sensing elements in the second configuration further comprises creating a preferential energy state for the subset of the plurality of aptamer sensing elements in the second configuration.

8. The method of claim 6, wherein the step of retaining the subset of the plurality of aptamer sensing elements in the second configuration further comprises binding together a complementary pair of linkers, wherein the complementary pair of linkers is attached to each aptamer sensing element.

9. The method of claim 6, wherein the step of retaining the subset of the plurality of aptamer sensing elements in the second configuration further comprises moving a plurality of functional groups into proximity so that a subset of the plurality of functional groups interact with each other, wherein the plurality of functional groups is attached to each aptamer sensing element.

10. The method of claim 6, wherein the step of retaining the subset of the plurality of aptamer sensing elements in the second configuration further comprises attaching one or more blockers to each aptamer sensing element, wherein the one or more blockers detaches when the selected aptamer sequence interacts with the target analyte.

11. The method of claim 6, wherein the step of retaining the subset of the plurality of aptamer sensing elements in the second configuration further comprises using a hydrophobic structure to attract a hydrophobic redox moiety and using a hydrophilic structure to attract a hydrophilic aptamer.

12. The method of claim 6, wherein the step of generating an aggregated signal from the plurality of aptamer sensing elements further comprises the steps of: interrogating an electrode to generate an electrical signal from the plurality of aptamer sensing elements, and retaining a subset of the plurality of aptamer sensing elements in the second configuration over multiple interrogation cycles to produce an aggregated signal for comparison to a signal threshold.

13. The method of claim 6, further comprising measuring a time-to-threshold, there the time-to-threshold is a time interval between an earlier first time and a later second time, wherein the first time represents when the EAB sensor begins sensing biofluid, and the second time represents when the aggregated signal reaches a first signal threshold.

14. The method of claim 13, further comprising measuring a time interval between an earlier third time and a later fourth time, wherein the third time represents when the aggregated signal reaches the first signal threshold; and where the fourth time represents when the aggregated signal reaches a second signal threshold.

15. The method of claim 13, further comprising using the time-to-threshold to estimate a concentration value for the target analyte in the biofluid.

16. The method of claim 15, further comprising using a biofluid flow rate to estimate a concentration value for the target analyte in the biofluid.
